(19) United States
(12) Patent Application Publication
(10) Pub. No.: US 2019/0015638 A1
(43) Pub. Date: Jan. 17, 2019

(54) POROUS BALLOON HAVING RADIOPAQUE MARKER

(71) Applicants: Boston Scientific Scimed, Inc., Maple Grove, MN (US); Mayo Foundation for Medical Education and Research, Rochester, MN (US)

(72) Inventors: Sarah M. Gruba, Vadnais Heiohts, MN (US); James A. Klos, Bay City, WI (US); James P. Rohl, Prescott, WI (US); Samuel J. Asirvatham, Rochester, MN (US); Suraj Kapa, Rochester, MN (US); Douglas Pennington, Stillwater, MN (US)

(21) Appl. No.: 16/035,869

(22) Filed: Jul. 16, 2018

Related U.S. Application Data
(60) Provisional application No. 62/533,497, filed on Jul. 17, 2017.

(51) Int. Cl. A61M 25/10; A61B 90/00

(52) U.S. Cl. CPC A61M 25/1002; A61B 90/39; A61M 25/1027; A61B 2090/3966; A61M 2025/1086; A61M 25/104; A61M 2025/1079

(57) ABSTRACT

The present disclosure relates to medical devices that comprise (a) a balloon structure having a proximal end, a distal end, a porous region, a non-porous region and an interior chamber, and (b) one or more radiopaque markers disposed on the balloon structure, the radiopaque markers including a polymeric material and a radiopaque material. The present disclosure also relates to methods of forming such medical devices, systems that include such medical devices, and methods of using such medical devices and systems.

### CROSS-REFERENCE TO RELATED APPLICATIONS

This application claims the benefit of U.S. Provisional Application No. 62/533,497, entitled “POROUS BALLOON HAVING RADIOPAQUE MARKER,” filed Jul. 17, 2017, which is hereby incorporated by reference in its entirety.

### FIELD

This disclosure relates to porous balloons with radiopaque markers.

### BACKGROUND

Polymeric balloons are used in a number of medical products including balloon catheters. It is beneficial to provide such balloons with features that render them visible under radiographic imaging. Unfortunately, due to their flexibility, polymeric balloons do not readily lend themselves to the application of metal radiopaque markers.

Accordingly, there is a continuing need in the art for improvements in radiopaque markers for balloons.

### SUMMARY

In accordance with various aspects, the present disclosure pertains to medical devices that comprise (a) a balloon structure comprising a proximal end, a distal end, a porous region, a non-porous region and an interior chamber, and (b) one or more radiopaque markers disposed on the balloon structure, the radiopaque markers comprising a polymeric material and a radiopaque material. In certain embodiments, the balloon structure may comprise an electrospun balloon, among other possibilities.

In certain embodiments, which may be used in conjunction with any of the preceding aspects and embodiments, the polymeric material may comprise an elastomeric material, for example, a silicone material, among other possibilities.

In certain embodiments, which may be used in conjunction with any of the preceding aspects and embodiments, the one or more radiopaque markers may be formed by a process that comprises applying a solidifiable material, which comprises the radiopaque material and is in liquid form, to a surface of the balloon structure, after which the solidifiable material is solidified to form the one or more radiopaque markers. For example, the solidifiable material may be curable material that is solidified during a curing process, or the solidifiable material may be a thermoplastic polymer melt that is solidified upon cooling, among other possible solidification processes.

In certain embodiments, which may be used in conjunction with any of the preceding aspects and embodiments, the polymeric material may be a room temperature curable adhesive. The room temperature curable adhesive may comprise, for example, a polysiloxane having acetoxy groups, among other possibilities.

In certain embodiments, which may be used in conjunction with any of the preceding aspects and embodiments, the polymeric material may be a UV curable adhesive. The UV curable adhesive may comprise, for example, a free-radical-generating photoinitiator, a multifunctional unsaturated oligomer and, optionally, an unsaturated oligomer, or the UV curable adhesive may comprise, for example, a cationic photoinitiator and an epoxide compound, among other possibilities.

In certain embodiments, which may be used in conjunction with any of the preceding aspects and embodiments, the one or more radiopaque markers may define one or more boundaries between the porous region and the nonporous region. For instance, the porous region may be in the form of a porous band having a proximal boundary and a distal boundary, in which case, (a) one or more radiopaque markers may be provided at the proximal boundary, (b) one or more radiopaque markers may be provided at the distal boundary, or (c) one or more radiopaque markers may be provided at the proximal boundary, and one or more radiopaque markers may be provided at the distal boundary.

In certain embodiments, which may be used in conjunction with any of the preceding aspects and embodiments, one or more radiopaque markers may mark the proximal end of the balloon structure.

In certain embodiments, which may be used in conjunction with any of the preceding aspects and embodiments, one or more of radiopaque markers may form a first band positioned at the proximal end of the balloon and/or one or more radiopaque markers may form a second band positioned at the distal end of the balloon.

In certain embodiments, which may be used in conjunction with any of the preceding aspects and embodiments, a plurality of equally spaced radiopaque markers of equal length in the form a first band may be positioned at the proximal end of the balloon and a plurality of equally spaced radiopaque markers of equal length in the form a second band may be positioned at the distal end of the balloon.

In certain embodiments, which may be used in conjunction with any of the preceding aspects and embodiments, the medical devices may further comprise an elongate body, and the balloon structure may be positioned at a distal end of the elongate body. In certain of these embodiments, the elongate body may comprise a lumen in fluid communication with the interior chamber that is configured to supply fluid to the interior chamber such that the fluid permeates through the porous region of the balloon structure. In certain of these embodiments, the medical devices may further comprise an additional interior chamber and the elongate body may comprise an additional lumen in fluid communication with the additional interior chamber, in which case the lumen may be configured to supply a first fluid to the interior chamber of the balloon structure such that the first fluid permeates through the porous region of the balloon structure, and the additional lumen may be configured to supply a second fluid to the additional interior chamber of the balloon structure such that the second fluid inflates the balloon structure.

In certain embodiments, which may be used in conjunction with any of the preceding aspects and embodiments, the medical devices may further comprise an electrode positioned within the interior of the balloon structure. In certain of these embodiments, the medical devices may further comprise a tip electrode that is configured to form a ground or a closed-loop with the electrode positioned within the interior of the balloon structure.

In certain embodiments, which may be used in conjunction with any of the preceding aspects and embodiments, the medical devices may be irreversible electroporation (IRE) devices.

In other aspects, the present disclosure is directed to systems that comprise (a) a medical device in accordance with any of the preceding aspects and embodiments and (b) a controller configured to supply electrical energy to the medical device. For example, the controller may be configured to supply DC energy, RF energy, or both, to the medical device.

Details of various aspects and embodiments of the disclosure are set forth in the description to follow and in the accompanying drawings. Other features and advantages of the disclosure will be apparent from the description and drawings, and from the claims.

### BRIEF DESCRIPTION OF THE DRAWINGS

FIG. 1 is a schematic cutaway view of a distal end of a catheter that comprises a single-chamber porous balloon structure having radiopaque markers, in accordance with an embodiment of the present disclosure.

FIG. 2 is a schematic cutaway view of a distal end of a catheter that comprises a dual-chamber porous balloon structure having radiopaque markers, in accordance with an embodiment of the present disclosure.

FIG. 3 is a schematic cutaway view of a distal end of a catheter that comprises a dual-chamber porous balloon structure having radiopaque markers, in accordance with another embodiment of the present disclosure.

FIG. 4 is a schematic cutaway view of a distal end of a catheter that comprises a three-chamber porous balloon structure having radiopaque markers, in accordance with an embodiment of the present disclosure.

FIG. 5A is a photograph of an apparatus in accordance with the present disclosure.

FIG. 5B is a radiographic image of the apparatus of FIG. 5A.

FIG. 6A is a schematic view of a distal end of a catheter, partially positioned in a vein and partially positioned in an atrium, in accordance with an embodiment of the present disclosure.

FIG. 6B is a schematic view of a distal end of a catheter, positioned entirely in a vein, in accordance with an embodiment of the present disclosure.

FIG. 7A is a schematic view of a distal end of a catheter, partially positioned in a vein and partially positioned in an atrium, in accordance with an embodiment of the present disclosure.

FIG. 7B is a schematic view of a distal end of a catheter, positioned entirely in a vein, in accordance with an embodiment of the present disclosure.

### DETAILED DESCRIPTION

In various aspects, the present disclosure pertains to medical devices that comprise (a) an elongate body, (b) a balloon structure having a proximal end, a distal end, a porous region, a non-porous region, and at least one interior chamber positioned at a distal end of the elongate body, and (c) and at least one radiopaque marker disposed on the balloon structure, which comprises a polymeric material and a radiopaque material.

Balloon structures for use in accordance with the present disclosure may be formed from a variety of materials including the following, including combinations thereof, among others: polyurethanes, including thermoplastic polyurethanes, for example, polycarbonate-based polyurethanes (e.g. BIONATE, CHRONOFLEX, etc.), polyether-based polyurethanes, polyester-based polyurethanes, polyether- and polyester-based polyurethanes (e.g. TECOTHANE, PELLET HANE, etc.), polyisobutylene-based polyurethanes, and polysiloxane-based polyurethanes, among others; styrene-alkylene block copolymers, including styrene-isobutylene block copolymers such as poly(styrene-b-isobutylene-b-styrene) (SIBS) tri-block copolymers and styrene-isoprene-butadiene block copolymers, among others; fluoropolymers, including polyvinylidene fluoride (PVDF), poly(vinylidene fluoride-co-hexafluoropropene) (PVDF-HFP), and polytetrafluoroethylene (PTFE), among others; polyesters, including non-biodegradable polyesters such as polyethylene terephthalate and biodegradable polyesters such as polycaprolactone (PCL) and poly(lactic-co-glycolic acid) (PLGA), among others; and polyamides, including nylon (e.g., nylon 6) and polyether block amides, among others.

Balloons having porous and nonporous regions may be provided by any method known in the art. In certain beneficial embodiments, such balloons may be formed in conjunction with a fiber-forming process such as electrospinning, force spinning or melt-blowing, among other possible processes. Electrospinning is a process that uses electrical charge to create polymer fibers from a polymer-containing liquid (e.g., a polymer solution or polymer melt), Force spinning is a process that uses centrifugal force to create fibers. Melt-blowing is a process in which a polymer melt is extruded through a die and then stretched and cooled with high-velocity air to form fibers.

Solvents for forming polymer solutions for spinning processes such as electrospinning or force spinning will depend on the polymer that is in solution and include, for example, acetone, acetonitrile, heptane, dimethyl-formamide (DMF), dirnethylacetamide (DMAC), ethanol, ethyl acetate, methanol, 1-propanol, 2-propanol, tetrahydrofuran (THF), toluene, xylene, and combinations thereof, among others, Typical voltages for electrospinning range between 5000-30000 volts, among other possibilities.

In certain embodiments, polymer fibers may be formed into an interior cavity of a balloon-shaped mold or onto an exterior surface of a balloon-shaped mold using a suitable fiber forming process (e.g., an electrospinning process, etc.), or preformed polymer fibers may be placed into an interior cavity of a balloon-shaped mold or onto an exterior surface of a balloon-shaped mold. The mold may be formed from a removable material, for example, a material that may subsequently be melted or dissolved. In certain embodiments, polymer fibers are formed onto an external surface of a balloon-shaped mold that is formed of ice.

Once fibers are assembled in the shape of a balloon (e.g., while still remaining in or on a mold, or after removal from a mold), a curable liquid material such as a liquid room temperature curable material, a liquid thermoset material or a liquid UV curable material, e.g. a curable polydimethylsiloxane (PDMS) material, among many others, or a thermoplastic melt, may be applied to the fibers in those areas where it is desired to establish one or more nonporous regions. Upon curing (in the case where a curable material is employed) or cooling (in the case where a thermoplastic melt is employed), a balloon having porous and nonporous regions is produced.

In one particular example, a UV curable adhesive such as Med-1515 RTV silicone room temperature adhesive, available from NuSil™ Technology LLC, Carpinteria, Calif., may be applied to the fibers to plug up small gaps in the fibrous structure, thereby creating one or more non-porous regions. The adhesive may be non-diluted or diluted with heptane or xylene. Adhesive:solvent mass/mass dilution levels may range, for example, from 3:1 to 1:5 (e.g., 3:1, 2:1, 1:1, 1:2, 1:3, 1:4 or 1:5), among other values.

In an alternative process, a curable liquid material or a thermoplastic melt may be applied to an interior cavity of a balloon-shaped hollow mold or onto an exterior surface of a balloon-shaped internal mold in those areas where nonporous regions are desired. While the material remains at least partially in liquid form (e.g., where the thermoset material is uncured or only partially cured or where the thermoplastic material is held at or above its melting point), polymer fibers may be applied onto the material, for example, using an electrospinning process or an alternative process, Because the material is at least partially in liquid form, at least a portion of the polymer fibers penetrates into material. Upon curing (in the case where a curable material is employed) or cooling (in the case where a thermoplastic melt is employed), a balloon having porous and nonporous regions is produced.

Using the above and other methods, a balloon structure having a proximal end, a distal end, a porous region, a nonporous region and at least one interior chamber is formed.

Regardless of the method of forming the balloon structure, in various aspects of the present disclosure, at least one radiopaque marker is provided on the balloon structure by applying a solidifiable material, which comprises a suitable radiopaque material and is in liquid form, to a surface of the balloon structure. The solidifiable material may be applied to the porous region of the balloon structure, the nonporous region of the balloon structure, or both.

Among other locations, the solidifiable material may be applied, for example, boundaries lying between porous region(s) and nonporous region(s) the balloon, in order to form one or more radiopaque marker that delineate such boundaries. Among many other possible forms, the porous region(s) may comprise, for example, one or more porous bands that extend around a circumference of the balloon structure. In such cases, the solidifiable material may be applied, for example, in the form of one or more bands, in the form of a series of dots, in the form of a series of band segments (e.g., a series of arcs), or in another form, (a) to a region of the balloon structure that lies adjacent to the one or more porous bands, (b) to a region of the balloon structure that does not lie adjacent to the one or more porous bands, (c) to a portion (but not all) of a surface of the one or more porous bands, or (d) to a combination of the foregoing. Alternatively or in addition, the solidifiable material may be applied, for example, at a proximal and/or distal end of the balloon in order to form one or more radiopaque markers defining the same.

With specific reference to electrospun balloons, it is noted that such devices can be used for a variety of medical products. However, in many instances, due to their elasticity and/or the existence of porous regions, electrospun balloons do not lend themselves to the application of metal radiopaque markers that will dictate where areas of interest on the device are located. In addition, electrospun balloons are frequently porous throughout the thickness of the walls, which is not conducive to filling them with contrast for visualization purposes.

One particular application where it is beneficial to image particular locations on an electrospun balloon, is when the electrospun balloon is used in conjunction with irreversible electroporation (IRE). In IRE, one or more porous areas of the electrospun balloon are placed in the vicinity of tissue that is being treated. Without radiopaque markers, it is difficult to know if the one or more porous areas of the balloon are in the correct location of the anatomy. Unlike radiofrequency energy or thermal injury from DC ablation, IRE does not require contact. Rather, it works by having an overlying electrical field cause electroporation of a cell membrane, and subsequent cell death. Because the electric field may concentrate with higher field strength at areas of abrupt impedance differences (which may be the result of tissue characteristics, interfaces with the blood, etc.), it may be describable to use radiopaque markers comprising a polymeric material and a radiopaque material as described herein to identify those areas where increasing field strength may be needed due to a varying impedance.

It is also beneficial to know when the proximal end of the balloon (and thus the entire balloon) is out of the catheter. Contrast can normally be used to identify the proximal end of the balloon. However, contrast can leak into the body through the one or more porous areas. Without the use of contrast, at least one radiopaque marker is useful to define the proximal end of the balloon.

In certain applications, it may be beneficial to know the location of a balloon during procedures for the treatment of atrial fibrillation. In this regard, during these procedures, it may be beneficial to know whether a balloon is within a vein (e.g., a pulmonary vein) or is free in the atrium. For certain balloon designs, it may be beneficial to know whether or not the balloon is opposed to an ostial left atrial wall. In addition, beyond IRE, it may beneficial to know whether a balloon is properly positioned to obstruct flow in a blood vessel (e.g., in a procedure where radiofrequency ablation is conducted using an electrode positioned outside the pulmonary vein).

In these and other applications, a balloon may be provided in which a plurality of radiopaque markers form one or more lines of equally spaced markers that encircle the balloon (e.g., in the form of a single band, or in the form of two, three, four, five, six or more bands that are offset form one another along an axial length of the balloon). When the balloon is expanded in an unobstructed space, the radiopaque markers expand and separate equidistant from each other and with equal radial distance from the axis. When a part of the balloon is in a vein and a part is in an ostium or an atrium, then this relationship will no longer apply with the markers expanding and separating relative to one another to a lesser extent in the vein and a greater extent in the ostium or atrium. This information is useful, for example, because the expansion and separation of the radiopaque markers informs the healthcare provider as to which part of the balloon (and thus which electrodes) is inside the vein, which part of the balloon (and thus which electrodes) is outside the vein, as well as whether or not the vein is being occluded by the balloon.

Radiopaque markers in accordance with the present disclosure can be applied anywhere on the surface of a given device and can be used for many different iterations of devices that require markers, including balloon devices in which a solid metal band cannot be placed around the balloon without damaging the device. By adding radiopaque markers in accordance with the present disclosure one can see the location of the relevant parts of the device, for example, under fluoroscopy.

In various embodiments, one or more radiopaque markers are placed alongside a balloon's porous areas to show where the tissue is being treated. One or more radiopaque markers may also be placed at the proximal end of the balloon to ensure the entire balloon is out of the catheter when deployed. In various embodiments, radiopaque markers in accordance with the present disclosure (e.g., markers that comprise a suitable radiopaque material dispersed in an elastomeric material such as a silicone/polysiloxane material, among others), are able to expand and contract to adjust for the various sizes of the balloon during inflation and deflation. In addition, radiopaque markers in accordance with the present disclosure are readily applied to a given device, and do not significantly impact the shape of device. Where a radiopaque marker is formed from a solidifiable adhesive material, it can also be used to bind two areas of the device (e.g., used to bind a balloon to a catheter, or to bind an inner balloon to an outer balloon, if a radiopaque marker is desired in that location).

Radiopaque materials suitable for use in conjunction with the present disclosure include radiopaque metals and radiopaque metal compounds, such as those that contain barium, bismuth, cerium, tungsten, tantalum, indium, gold, or platinum, among other metals. Particular examples of radiopaque metal compounds include barium sulfate, bismuth trioxide, bismuth subcarbonate, bismuth oxychloride, or cerium oxide, among others. Radiopaque materials also include polymeric materials that comprise iodine or bromine in the polymer structure.

Solidifiable materials include any suitable solidifiable polymer material known in the medical device art. In some embodiments, the solidifiable material is a medically acceptable adhesive material and may be, for example, a room temperature curable adhesive material or a UV curable adhesive material.

In certain embodiments, the solidifiable materials may be diluted with a suitable solvent.

In certain embodiments, the solidifiable materials may comprise from 5-75% by weight (e.g., 5, 10, 15, 20, 25, 30, 35, 40, 45, 50, 55, 60, 65, 70 or 75% by weight) of a radiopaque material.

Particular examples of room temperature curable adhesives include those that comprise a polymer (e.g., a polysiloxane) having reactive groups. In particular embodiments, a reactive polymer in the form of a polysiloxane (e.g., a polydimethylsiloxane having acetoxy groups) may be employed. In the case of a polysiloxane having acetoxy groups, and without wishing to be bound by theory, upon exposing the reactive polymer to ambient moisture, the acetoxy groups are hydrolyzed to yield silanols, which further condense to link the polymer chains together. The curing process can be accelerated by curing at elevated temperatures, by curing at elevated humidity, or both. In addition, a silanol terminated polysiloxane may be cross-linked, for example, using triacetoxymethylsilane or triacetoxyethylsilane in the presence of a suitable catalyst, among other alternative processes. Other examples of room temperature curable adhesives include room temperature curable epoxy adhesives (e.g., EP21BAS, a two component, radiopaque, epoxy system for bonding, available from Master Bond, Hackensack, N.J., USA).

Examples of UV curable adhesives include UV curable adhesive materials that contain a free radical generating photoinitiator and a compound having multiple unsaturated groups (e.g., acrylate, methacrylate or vinyl groups), such as an oligomer having multiple unsaturated groups and, optionally, a monomer having multiple unsaturated groups. Specific examples of free radical generating photoinitiators include, for example, type I or type II photoinitiators, such as benzoin ether, 1-hydroxy-cyclohexylphenyl-ketone or benzophenone, among others. Specific examples of oligomers having multiple unsaturated groups include acrylate oligomers such as epoxy acrylates (e.g., bisphenol-A-epoxy acrylate), aliphatic urethane acrylates (e.g., IPDI-based aliphatic urethane acrylates), aromatic urethane acrylates, polyether acrylates, polyester acrylates, aminated acrylates, and acrylic acrylates. Specific examples of monomers include mono- di- and tri-functional monomers such as trimethylolpropane triacrylate, propoxylated trimethylolpropane triacrylate, ethoxylated trimethylolpropane triacrylate, tripropylene glycol diacrylate, hexanediol diacrylate, isobornyl acrylate, isodecyl acrylate, ethoxylated phenyl acrylate, and 2-phenoxyethyl acrylate, among others.

Further examples of UV curable adhesives include UV curable adhesive materials that comprise a cationic photoinitiator and an epoxide compound. Particular examples of cationic photoinitiators include onium salts such as aryl sulfonium and aryl iodonium salts. Specific examples of epoxide compounds include cycloaliphatic epoxide compounds and aromatic epoxide compounds such as 3,4-epoxy-cyclohexylmethyl-3,4-epoxy-cyclohexane-carboxylate and Bisphenol A diglycidyl ether, and polysiloxanes having epoxy groups, among others.

In some particular embodiments, a room temperature curable adhesive (e.g., Med-1515 RTV silicone room temperature adhesive) is admixed with a radiopaque material (e.g., barium powder, etc.) and applied to a balloon structure to create one or more radiopaque markers. For example, the admixture may be applied to non-porous areas (e.g., at a proximal end of the balloon) or at the edges of porous areas. The adhesive may be non-diluted or diluted with a suitable solvent (e.g., heptane, xylene, etc.). Adhesive:solvent mas/mass dilution levels may range, for example, from 3:1 to 1:5 (e.g., 3:1, 2:1, 1:1, 1:2, 1:3, 1:4 or 1:5), among others values. The adhesive may then be allowed to cure at room temp overnight or in an oven at elevated humidity.

In some particular embodiments, a UV curable adhesive may be admixed with a radiopaque material (e.g., barium powder, etc.) and applied to a balloon to create radiopaque markers. For example, the admixture may be applied to non-porous areas (e.g., at a proximal end of the balloon) or at the edges of porous areas. The adhesive may be non-diluted or diluted with a suitable solvent, if appropriate. The adhesive may then be cured by exposure to UV light of a suitable wavelength for a suitable time, depending on the particular adhesive selected.

In certain embodiments, the balloon structures described herein may be provided in conjunction with devices in which electrical energy is delivered, for example, an irreversible electroporation (IRE) balloon device, in which one or more electrodes are positioned within an interior of the balloon structure.

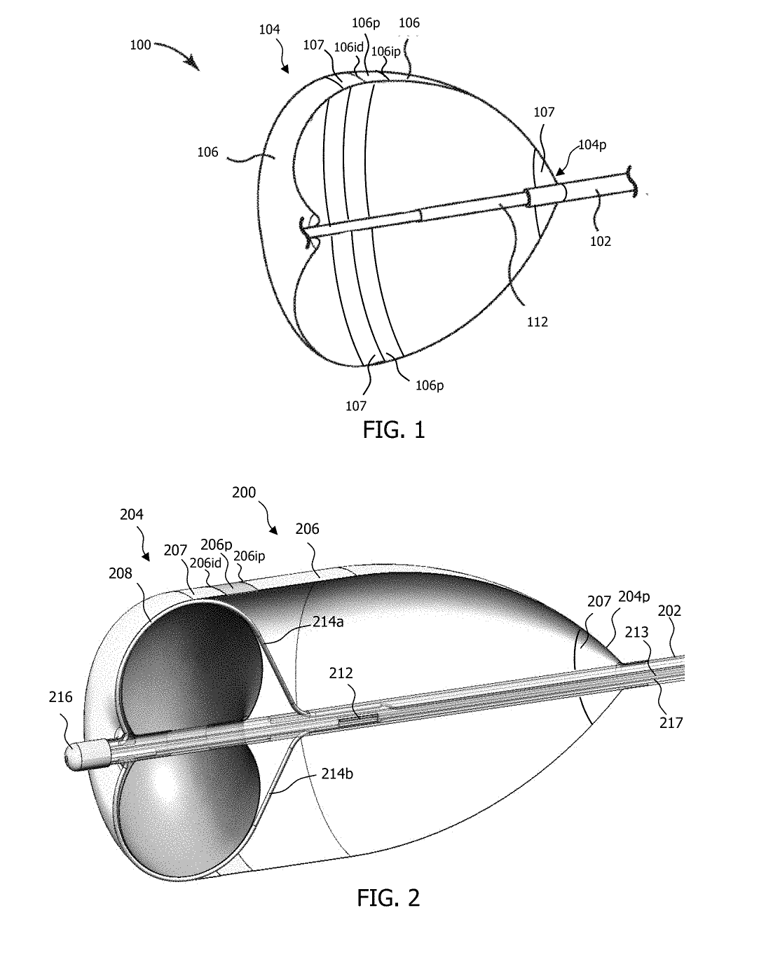

In this regard, FIG. 1 shows a cutaway illustration of an exemplary apparatus 100 for applying ablation therapy to a tissue region in accordance with embodiments of the disclosure. The apparatus 100 includes a catheter having an elongate body 102. At or near a distal portion of the elongate body 102 is a balloon structure 104. The balloon structure 104 may be attached to or formed on the elongate body 102.

The balloon structure 104 may include a first portion 106, at least a section of which has a first permeability. The balloon structure 104 is configured to inflate in response to a liquid inflation medium being provided thereto. Moreover, the first portion 106 of the balloon structure 104 may be configured to permeate the liquid therethrough in response to inflation of the balloon structure 104 (the liquid may be, for example, saline, or a pharmacological agent, etc.) while at the same time anchoring the elongate body 102 at a tissue region.

For example, the balloon structure 104 may include a porous region 106p in the first portion 106 that is permeable to liquid, while a remainder of the first portion 106 is substantially impermeable to liquid. Thus, at least a portion 106p of the balloon structure 104 may be permeable.

The balloon structure 104 may be positioned at a target tissue region for ablation. The balloon structure 104 may be configured to deploy within the vessel such that the porous region 106p is adjacent the vessel wall. The first portion 106 may permeate the liquid to the tissue region (e.g., the vessel wall) through section 106p.

In accordance with the present disclosure, the balloon structure 104 is also provided with one or more radiopaque markers 107 that comprise a polymeric material and a radiopaque material. One or more markers 107 may be disposed at the proximal edge 106ip and/or the distal edge 106id of the porous region 106p (in this case a marker 107 in the shape of a band is disposed at the distal edge 106id of the porous region 106p). In addition one or more radiopaque markers 107 may be disposed at a proximal end 104p of the balloon structure.

The apparatus 100 may also include one or more electrodes configured to deliver energy to a tissue region. As shown in FIG. 1, the apparatus 100 includes an electrode 112 arranged within the balloon structure 104. In certain instances, the electrode 112 may be arranged within the first portion 106 and configured to deliver energy in response to a direct current applied thereto. The energy from the electrode 112 may be applied through an external surface of the first portion 106 of the balloon structure 104 by an electric field generated by an external source/controller (not shown) and transferred through a wire within the elongate body 102. The electric energy can be transmitted to the tissue region (e.g., the vessel wall) via the liquid that exudes from through porous region 106p of the first portion 106 of the balloon structure 104. The electric field may at least partially cause apoptotic cell death and/or necrosis to the tissue receiving the energy. In certain instances, while an electric field for ablation is being applied, transmission of the liquid through section 106p of the first portion 106 of the balloon structure 104 to the tissue can be continued. In this regard, an electric field may be applied while continuously pumping liquid into the balloon or may be applied while flow of fluid into the balloon is ceased for a short period of time, during which the liquid continues to leak from the balloon due to the residual pressure in the balloon.

In certain instances and as noted above, the electric field may be generated by applying direct current to the electrode 112. The use of direct current may cause apoptotic cell death to the tissue receiving the ablation energy. The direct current may form pores in the cells of the tissue region such that are irreversible (e.g., the pores do not close). The balloon structure 104 being adjacent the tissue may provide controlled and direct ablation of a target area while mitigating against down-stream proliferation of the ablation energy.

Another embodiment is illustrated in FIG. 2, which shows a cutaway illustration of another exemplary apparatus 200 for applying ablation therapy to a tissue region in accordance with the disclosure. The apparatus 200 includes a catheter having an elongate body 202. At or near a distal portion of the elongate body 202 is a balloon structure 204. The balloon structure 204 may be attached to or formed on the elongate body 202.

The balloon structure 204 may include a first portion 206 of that forms a first chamber and a second portion 208 that forms a second chamber. The first portion 206 may be deposited or attached onto the second portion 208. The balloon structure 204 may include a porous region 206p in the first portion 206 that is permeable to liquid, while a remainder of the first portion 206 may be substantially impermeable to liquid. The second portion 208 may be substantially impermeable to liquid. The balloon structure 204 may be configured to inflate in response to an inflation medium being provided thereto. In certain instances, the first portion 206 and the second portion 208 may be inflated using a single inflation medium, or the first portion 206 and the second portion 208 may be separately inflated using a first inflation medium and a second inflation medium. As a result, in certain instances, the first portion 206 of the balloon structure 204 may be configured to permeate a liquid therethrough in response to inflation of the balloon structure 204 (the liquid may be, for example, saline, a pharmacological agent, etc.) and the second portion 208 of the balloon structure 204 may be configured to anchor the elongate body 202 at a tissue region.

The balloon structure 204 may be positioned at a target tissue region for ablation. The balloon structure 204 may be configured to deploy within the vessel such that porous region 206p is adjacent the vessel wall. The porous region 206p may permeate the liquid to the tissue region (e.g., the vessel wall). In addition, the second portion 208 may be configured to anchor the elongate body 202 at the tissue region.

In accordance with the present disclosure, the balloon structure 204 is also provided with one or more radiopaque markers 207 that comprise a polymeric material and a radiopaque material. One or more markers 207 may be disposed at the proximal edge 206ip and/or the distal edge 206id of the porous region 206p (in this case a marker 207 in the shape of a band is disposed at the distal edge 206id of the porous region 206p). In addition, one or more radiopaque markers 207 may be disposed at a proximal end 204p of the balloon structure.

The apparatus 200 may include one or more electrodes configured to deliver energy to a tissue region. As shown in FIG. 2, the apparatus includes an electrode 212 arranged within the balloon structure 204. In certain instances, the electrode 212 may be arranged within the first portion 206 and configured to deliver energy in response to a direct current applied thereto. The energy from the electrode 212 may be applied through an external surface of the first portion 206 of the balloon structure 204 by an electric field generated by an external source/controller (not shown) and transferred through a wire 213 within the elongate body 202. The electrical energy can be transmitted to the tissue region (e.g., the vessel wall) via the liquid that exudes from the porous region 206p of the first portion 206. The electric field may at least partially cause apoptotic cell death to the tissue receiving the energy. In certain instances while an electric field for ablation is being applied, transmission of the liquid from the porous region 206p of the first portion 206 of the balloon structure 204 to the tissue can be continued.

In certain instances and as noted above, the electric field may be generated by applying direct current to the electrode 212. The use of direct current may cause apoptotic cell death to the tissue receiving the ablation energy. The direct current may form pores in the cells of the tissue region such that are irreversible (e.g., the pores do not close). The balloon structure 204 being adjacent the tissue may provide controlled and direct ablation of a target area while mitigating against down-stream proliferation of the ablation energy.

The apparatus 200 may also include a tip electrode 216 that is configured to form a ground or a closed-loop with the electrode 212. Similar to the electrode 212, the tip electrode 216 may be coupled to the external source/controller via a wire 217 within the elongate body 202. The external source/controller may apply, for example, RF ablation energy or DC current. Thus, the tip electrode 216 may function as a single point ablation electrode when the external source/controller is configured to apply RF ablation energy.

In certain instances, the electrode 212 and/or the tip electrode 216 may also be configured to measure the localized intracardial electrical activity. The wire 213 and/or the wire 217 may also be electrically coupled to a mapping signal processor such that electrical events in myocardial tissue can be sensed for the generation of electrograms, monophasic action potentials (MAPs), isochronal electrical activity maps, and the like. The electrode 212 and/or the tip electrode 216 may allow the physician to measure the electrical activity of the tissue region (e.g., the lack of electrical activity indicates ablated tissue, whereas the presence of electrical activity indicates live tissue).

In some instances, the apparatus 200 may also include pacing electrodes 214a, 214b. The pacing electrodes 214a, 214b may be arranged within the balloon structure 204. The pacing electrodes 214a, 214b may be electrically coupled to a mapping signal processor such that electrical events in myocardial tissue can be sensed for the generation of electrograms, monophasic action potentials (MAPs), isochronal electrical activity maps, and the like. The pacing electrodes 214a, 214b may allow the physician to measure the electrical activity of the tissue region (e.g., the lack of electrical activity indicates ablated tissue, whereas the presence of electrical activity indicates live tissue). The ablation energy applied via the electrode 212 may be altered based on the electrical activity measured by the pacing electrodes 214a, 214b, which may be used to determine a target location for the ablation therapy.

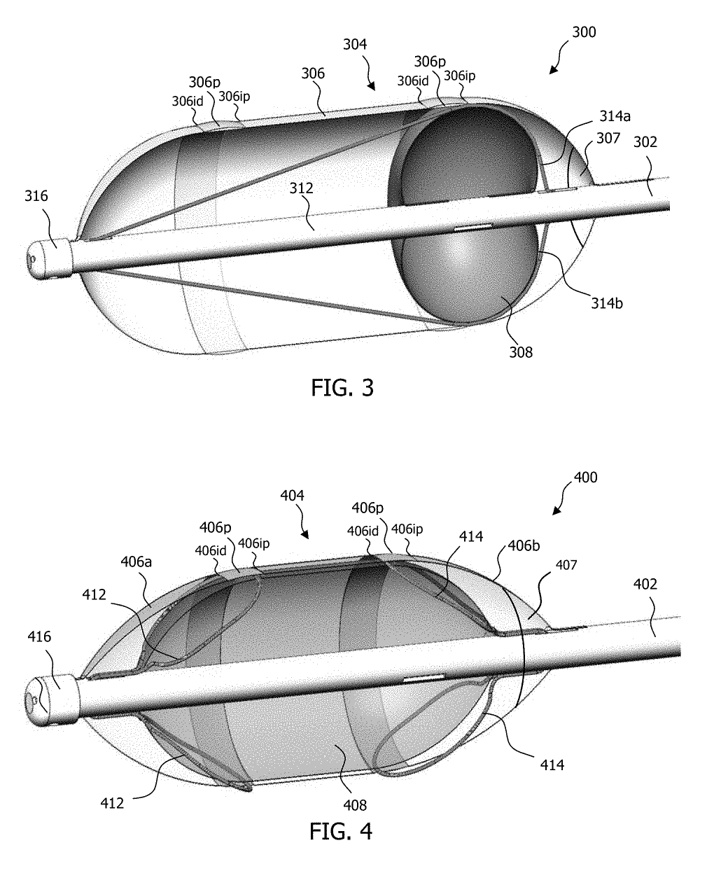

Another embodiment is illustrated in FIG. 3, which shows a cutaway illustration of an exemplary apparatus 300 for applying ablation therapy to a tissue region in accordance with embodiments of the disclosure. The apparatus 300 includes a catheter having an elongate body 302. At or near a distal portion of the elongate body 302 is a balloon structure 304.

Analogous to FIG. 2, balloon structure 304 of FIG. 3 may include a first portion 306 of that forms a first chamber and a second portion 308 that forms a second chamber. The balloon structure 304 may include two porous regions 306p in the first portion 306 that are permeable to liquid, while a remainder of the first portion 306 is substantially impermeable to liquid. The second portion 308 may be substantially impermeable to liquid. The balloon structure 304 may be configured to inflate in response to an inflation medium being provided thereto. In certain instances, the first portion 306 and the second portion 308 may be inflated using a single inflation medium, or the first portion 306 and the second portion 308 may be separately inflated using a first inflation medium and a second inflation medium. As a result, in certain instances, the first portion 306 of the balloon structure 304 may be configured to permeate a liquid therethrough in response to inflation of the balloon structure 304 (the liquid may be, for example, saline, a pharmacological agent, etc.) and the second portion 308 of the balloon structure 304 may be configured to anchor the elongate body 302 at a tissue region.

The balloon structure 304 may be positioned at a target tissue region for ablation. The balloon structure 304 may be configured to deploy within the vessel such that porous regions 306p are adjacent the vessel wall. The porous regions 306p may permeate the liquid to the tissue region (e.g., the vessel wall). In addition, the second portion 308 may be configured to anchor the elongate body 302 at the tissue region.

In accordance with the present disclosure, the balloon structure 304 is also provided with radiopaque markers 307 that comprise a polymeric material and a radiopaque material. One or more markers may be disposed at the proximal edge 306ip and/or the distal edge 306id of each porous region 306p (e.g., in the shape of a continuous or discontinuous band, not separately illustrated). In addition one or more radiopaque markers 307 may be disposed at a proximal end 304p of the balloon structure.

Analogous to FIG. 2, the apparatus 300 of FIG. 3 may include an electrode 312 arranged within the balloon structure 304, a tip electrode 316 that is configured to form a ground or a closed-loop with the electrode 312, and pacing electrodes 314a, 314b. These components may be operated in a fashion analogous to that described in conjunction with FIG. 2.

Still another embodiment is illustrated in FIG. 4, which shows a cutaway illustration of an exemplary apparatus 400 for applying ablation therapy to a tissue region in accordance with the present disclosure. The apparatus 400 includes a catheter having an elongate body 402. At or near a distal portion of the elongate body 402 is a balloon structure 404.

The balloon structure 404 of FIG. 4 may include a first portion 406a of that forms a first chamber, a second portion 408 that forms a second chamber, and a third portion 406b that forms a third chamber. The balloon structure 404 may include two porous regions 406p, one in the first portion 406a and another in the third portion 406b, which are permeable to liquid, while a remainder of the first and third portions 406a, 406b are substantially impermeable to liquid. The second portion 408 may be substantially impermeable to liquid. The balloon structure 404 may be configured to inflate in response to an inflation medium being provided thereto. In certain instances, the first portion 406a, the second portion 408, and the third portion 406b, may be inflated using a single inflation medium, or the first portion 406a, the second portion 408, and the third portion 406b may be separately inflated using separate inflation media. As a result, in certain instances, the first and third portions 406a, 406b of the balloon structure 404 may be configured to permeate a liquid therethrough in response to inflation of the balloon structure 404 (the liquid may be, for example, saline, a pharmacological agent, etc.) and the second portion 408 of the balloon structure 404 may be configured to anchor the elongate body 402 at a tissue region.

The balloon structure 404 may be positioned at a target tissue region for ablation. The balloon structure 404 may be configured to deploy within the vessel such that porous regions 406p are adjacent the vessel wall. The porous regions 406p may permeate the liquid to the tissue region (e.g., the vessel wall). In addition, the second portion 408 may be configured to anchor the elongate body 402 at the tissue region.

In accordance with the present disclosure, the balloon structure 404 is also provided with radiopaque markers 407 that comprise a polymeric material and a radiopaque material. One or more markers may be disposed at the proximal edge 406ip and/or the distal edge 406id of each porous region 406p (e.g., in the shape of a continuous or discontinuous band, not separately illustrated). In addition one or more radiopaque markers 407 may be disposed at a proximal end 404p of the balloon structure.

The balloon structure 404 of FIG. 4 may also include electrodes 412 in the first portion 406a, electrodes 414 in the third portion 406b, and a tip electrode 416. The electrode 412 may be configured to form a ground or a closed-loop with the electrode 414. Each of the electrodes 412, 414 may also be is configured to form a ground or a closed-loop with the tip electrode 416. These components may be operated in a fashion analogous to that described in conjunction with FIG. 2.

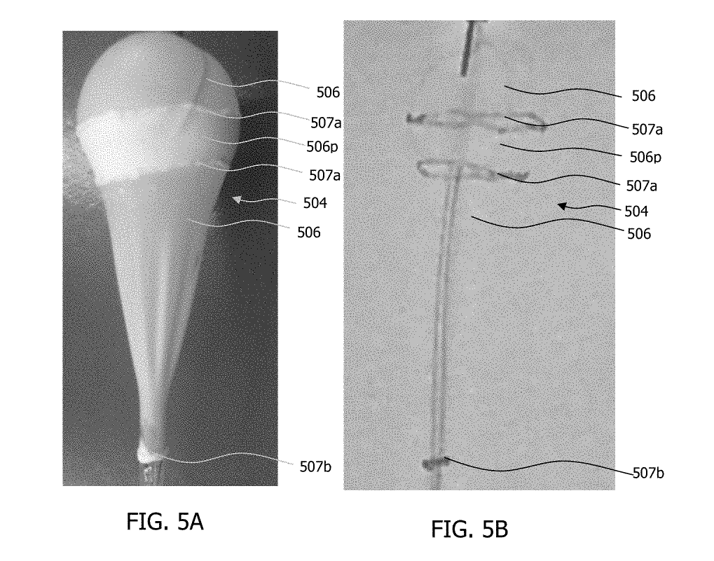

FIG. 5A is a photograph of an apparatus in accordance with the present disclosure that includes a catheter having a balloon structure 504, that includes a first portion 506 of that forms a first chamber and has a porous region 506p, two radiopaque markers 507a disposed at proximal and distal edges of the porous region 506p, and a single radiopaque marker 507b disposed at a proximal end of the balloon structure 504. FIG. 5B is a radiographic image of the balloon structure 504 when positioned in a subject and clearly shows the two radiopaque markers 507a that mark the boundaries of the porous region 506p and the single radiopaque marker 507b that marks the proximal end of the balloon structure 504.

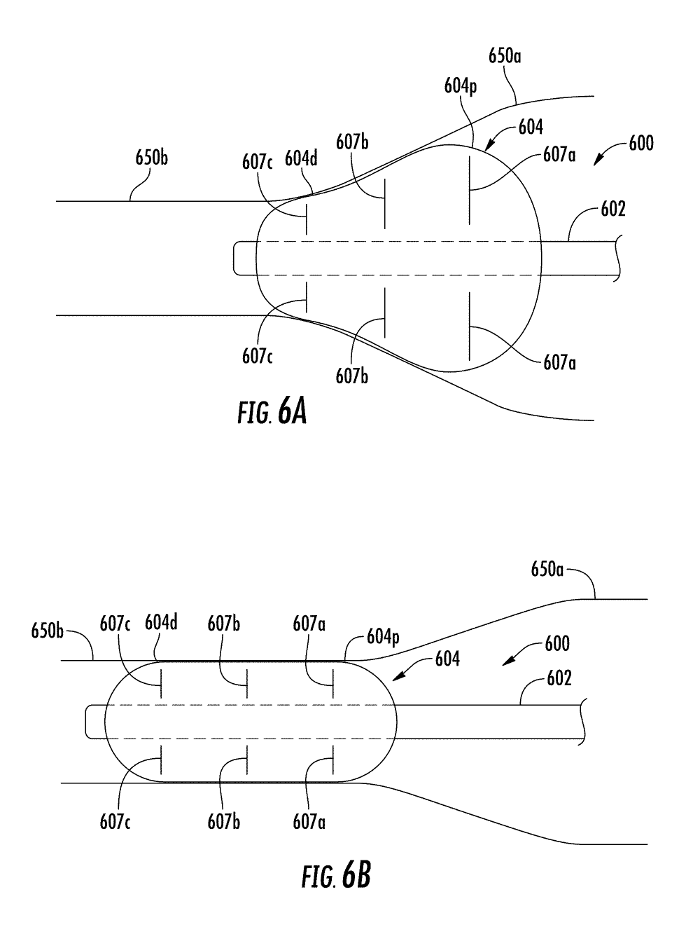

While the preceding embodiments illustrate radiopaque markers in the form of continuous bands, it may also be beneficial in those and other embodiments to replace a continuous radiopaque band with a discontinuous band, for example, in the form of a series of band segments, which may be, for example, in the form of arcs. FIGS. 6A and 6B show an exemplary apparatus 600 for applying ablation therapy to a tissue region in accordance with an embodiment of the disclosure. The apparatus 600 includes a catheter having an elongate body 602. At or near a distal end of the elongate body 602 is a balloon structure 604, which is formed of a compliant (e.g., elastomeric) material. On the balloon structure 604 are provided three groups of radiopaque markers 607a, 607b, 607c, each group encircling the balloon structure 604 around a longitudinal axis of the balloon structure 604. The radiopaque markers 607a, 607b, 607c are formed from an elastomeric material in the embodiment shown, allowing them to expand. In the embodiment shown, a first group of radiopaque markers 607a, in the form of a series of equally spaced arcs of equal length, encircles the balloon structure 604 at a proximal end 604p of the balloon structure 604, a second group of radiopaque markers 607b, in the form of a series of equally spaced arcs of equal length, encircles the balloon structure 604 at a center of the balloon structure 604, and a third group of radiopaque markers 607c, in the form of a series of equally spaced arcs of equal length, encircles the balloon structure 604 at a distal end 604d of the balloon structure 604. As seen in FIG. 6A, when expanded in a subject, for example, such that the distal end 604d of the balloon structure 604 is expanded in a vein 650b and a proximal end 604p of the balloon structure 604 is expanded in an atrium 650a, the radiopaque markers 607a, 607b, 607c expand and separate more in the atrium 650a than in the vein 650b. On the other hand, when expanded in a subject such that the entire balloon structure 604 is expanded in a vein 650b, the radiopaque markers 607a, 607b, 607c expand and separate in a more consistent fashion as shown in FIG. 6B.

Although three groups of radiopaque markers 607a, 607b, 607c are shown in FIGS. 6A and 6B, in other embodiments, one, two, four, five, six, seven, eight, nine, ten or more groups may be provided. Moreover, while four radiopaque markers are provided in each group in FIGS. 6A and 6B, in other embodiments, two, three, five, six, seven, eight, nine, ten or more radiopaque markers may be provided within each group.

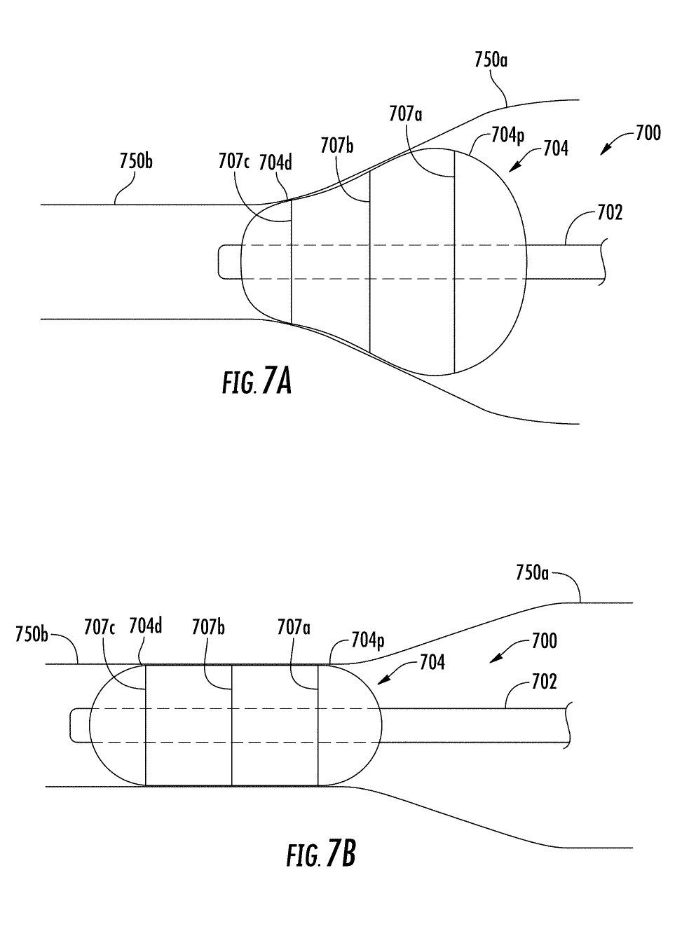

In contrast, FIGS. 7A and 7B show an exemplary apparatus 700 for applying ablation therapy to a tissue region in accordance with another embodiment of the disclosure. The apparatus 700 includes a catheter having an elongate body 702. At or near a distal end of the elongate body 702 is a balloon structure 704. On the balloon structure 704 are provided thee radiopaque markers 707a, 707b, 707c, each encircling the balloon structure 704 around a longitudinal axis of the balloon structure 704 in a continuous band. As above, the radiopaque markers 707a, 707b, 707c are formed from an elastomeric material in the embodiment shown, allowing them to expand along with the balloon structure 704. In the embodiment shown, a first radiopaque marker 707a encircles the balloon structure 704 at a proximal end 704p of the balloon structure 704, a second radiopaque marker 707b encircles the balloon structure 704 at a center of the balloon structure 704 and a third radiopaque marker 707c encircles the balloon structure 704 at a distal end 704d of the balloon structure 704. FIG. 7A shows an embodiment where the distal end 704d of the balloon structure 704 is expanded in a vein 750b and the proximal end 704p of the balloon structure 704 is expanded in an atrium 750a, in which case the radiopaque markers 707a, 707b, 707c expand to a larger diameter in the atrium 750a than in the vein 750b. On the other hand, when expanded in a subject such that the entire balloon structure 704 is expanded in a vein 750b, the radiopaque markers 707a, 707b, 707c expand in a more consistent fashion as shown in FIG. 7B. Thus, while distance between the radiopaque markers would not be an indicator in this embodiment (as compared with FIGS. 6A and 6B), the relative width and circular (or non-circular) nature of the expansion of the radiopaque markers may provide an indication with regard to whether or not the balloon structure is in the vein or the atrium.

While three circular radiopaque markers are provided in FIGS. 7A and 7B, in other embodiments, one, two, four, five, six, seven, eight, nine, ten or more circular radiopaque markers may be provided.

What is claimed is:

1. A medical device comprising (a) a balloon structure comprising a proximal end, a distal end, a porous region, a non-porous region and an interior chamber, and (b) one or more radiopaque markers disposed on the balloon structure, the radiopaque markers comprising a polymeric material and a radiopaque material.

2. The medical device of claim 1, wherein the balloon structure comprises an electrospun balloon.

3. The medical device of claim 1, wherein polymeric material comprises an elastomeric material.

4. The medical device of claim 1, wherein the one or more radiopaque markers are formed by a process that comprises applying a solidifiable material, which comprises the radiopaque material and is in liquid form, to a surface of the balloon structure, after which the solidifiable material is solidified to form the one or more radiopaque markers.

5. The medical device of claim 1, wherein the one or more radiopaque markers are formed from a room temperature curable adhesive and the radiopaque material.

6. The medical device of claim 5, wherein the room temperature curable adhesive comprises a polysiloxane having acetoxy groups.

7. The medical device of claim 1, wherein the one or more radiopaque markers are formed from a UV curable adhesive and the radiopaque material.

8. The medical device of claim 7, wherein the UV curable adhesive comprises a free radical generating photoinitiator, a multifunctional unsaturated oligomer and, optionally, a monomer having multiple unsaturated groups, or wherein the UV curable adhesive comprises a cationic photoinitiator and an epoxide compound.

9. The medical device of claim 1, wherein the one or more radiopaque markers indicate one or more boundaries between the porous region and the non-porous region.

10. The medical device of claim 9, (a) wherein the porous region is in a form of a porous band having a proximal boundary and a distal boundary and (b) at least one of the one or more radiopaque markers is provided at the proximal boundary, at least one of the one or more radiopaque markers is provided at the distal boundary, or both.

11. The medical device of claim 1, wherein at least one of the one or more radiopaque markers defines the proximal end of the balloon structure.

12. The medical device of claim 1, comprising at least one of the one or more radiopaque markers in a form a first band positioned at the proximal end of the balloon structure and at least one of the one or more radiopaque markers in a form a second band positioned at the distal end of the balloon structure.

13. The medical device of claim 12, wherein a plurality of equally spaced radiopaque markers of equal length form the first band and a plurality of equally spaced radiopaque markers of equal length form the second band.

14. A medical device comprising (a) a balloon structure comprising a proximal end, a distal end, a porous region, a non-porous region and an interior chamber, (b) one or more radiopaque markers disposed on the balloon structure, the radiopaque markers comprising a polymeric material and a radiopaque material, and (c) an elongate body, wherein the balloon structure is positioned at a distal end of the elongate body.

15. The medical device of claim 14, wherein the elongate body comprises a lumen in fluid communication with the interior chamber that is configured to supply fluid to the interior chamber such that the fluid permeates through the porous region of the balloon structure.

16. The medical device of claim 15, further comprising an electrode positioned within the interior chamber of the balloon structure.

17. The medical device of claim 16, further comprising a tip electrode that is configured to form a ground or a closed-loop with the electrode positioned within the interior chamber of the balloon structure.

18. The medical device of claim 16, wherein device is an irreversible electroporation (IRE) device.

19. A system comprising:
a medical device that comprises (a) a balloon structure comprising a proximal end, a distal end, a porous region, a non-porous region, an interior chamber, and an electrode positioned within the interior chamber, (b) one or more radiopaque markers disposed on the balloon structure, the radiopaque markers comprising a polymeric material and a radiopaque material, and (c) an elongate body, wherein the balloon structure is positioned at a distal end of the elongate body; and
a controller configured to supply electrical energy to the electrode.

20. The system of claim 19, wherein the controller is configured to supply DC or RF energy to the electrode.
